(19) United States
(12) Patent Application Publication
(10) Pub. No.: US 2020/0399343 A1
(43) Pub. Date: Dec. 24, 2020

(54) IMPROVED T CELL COMPOSITIONS AND METHODS

(71) Applicant: Allogene Therapeutics, Inc., South San Francisco, CA (US)

(72) Inventors: Javier Fernando CHAPARRO RIGGERS, San Mateo, CA (US); Thomas John VAN BLARCOM, Oakland, CA (US); Bijan Andre BOLDAJPOUR, San Francisco, CA (US)

(21) Appl. No.: 16/604,690

(22) PCT Filed: Apr. 18, 2018
(86) PCT No.: PCT/IB2018/052698

§ 371 Date: Oct. 11, 2019

Related U.S. Application Data
(60) Provisional application No. 62/487,215, filed on Apr. 19, 2017.

(51) Int. Cl. C07K 14/725; C07K 14/005; C07K 16/28; A61K 35/17; C12N 7/00; A61P 37/06; C12N 5/0783

(52) U.S. Cl. CPC C07K 14/7051; C07K 14/005; C07K 16/2803; A61K 35/17; C12N 7/00; A61P 37/06; C12N 2510/00; C07K 2319/03; C07K 2319/02; C07K 2317/622; C12N 2710/16122; C12N 2501/50; C12N 5/0636

(57) ABSTRACT

The present invention provides compositions and methods that downregulate major histocompatibility class I molecule cell surface expression, and uses of such compositions and methods for improving the functional activities of isolated T cells (e.g., gene-modified antigen-specific T cells, such as chimeric antigen receptor T (CAR-T) cells). In particular, the present invention provides methods and compositions for bolstering the therapeutic efficacy of CAR-T cells.

### CROSS-REFERENCE TO RELATED APPLICATIONS

This application claims priority to U.S. Provisional Application No. 62/487,215, filed on Apr. 19, 2017, which is incorporated by reference herein in its entirety.

### FIELD

The present invention relates generally to the use of immune cells (e.g., T cells) engineered to express a chimeric antigen receptor (CAR) to treat a disease.

### BACKGROUND

Chimeric antigen receptor T (CAR-T) cells have entered the clinic and have demonstrated very promising results (Maus, M. et al., 2014, Blood 123, 2625-35). Although the majority of subjects have been treated with autologous CAR-T cells which are derived from the subject's own T cells, allogeneic CAR-T cells derived from healthy donors offers a more commercially viable off-the-shelf option with the potential to treat a broader range of subjects.

Allogeneic CAR-T cells are generated by endowing T cells from healthy donors with CARs that are specifically activated by tumor associated antigens. Donor incompatibility could result in graft versus host (GvH) disease or elimination of the CAR-T cells through host versus graft (HvG) rejection as has been observed with allogeneic transplants. Rejection of the allogeneic T cells by the subject's immune system may limit the persistence of allogeneic CAR-T cells and lead to lower efficacy compared to autologous CAR-T cells (Berger, C. et al., 2015, Cancer Immunol Res 3, 206-16; Kochenderfer, J. et al., 2013, Blood 122, 4129-39). This may be particularly important in situations such as solid tumors, where long-term CAR-T persistence may be important for a durable response.

Thus, there is a need for allogeneic CAR-T cell having improved persistence.

### SUMMARY

The present invention provides compositions and methods that downregulate major histocompatibility class I (MHC Class I) cell surface expression, and uses of such compositions and methods for improving the functional activities of genetically modified T cells (e.g., gene-modified antigen-specific T cells, such as chimeric antigen receptor T (CAR-T) cells). In particular, the present invention provides methods and compositions for bolstering the therapeutic efficacy of CAR-T cells. While not to be bound by the theory, expression of viral protein leads to decreased MHC Class I cell surface expression results in decreased T cell recognition, which leads to increased in vivo persistence and consequently improved CAR-T cell efficacy

In one aspect, the invention provides an isolated T cell comprising a viral protein which decreases cell surface expression level of major histocompatibility complex (MHC) Class I relative to cell surface expression level of MHC Class I of an isolated T cell that does not comprise the viral protein, and a chimeric antigen receptor (CAR) comprising an extracellular ligand-binding domain, a transmembrane domain, and an intracellular signaling domain.

In some embodiments, the viral protein can be a human cytomegalovirus (hCMV) protein, adenovirus protein, herpesvirus protein, or human immunodeficiency virus protein. In some embodiments, the viral protein can be BFP, ICP47, K3, K5, E19, US3, US6, US2, U21, Nef, US10, or U21. In some embodiments, the viral protein can be K5. In some embodiments, the isolated T cell does not express any detectable MHC Class I molecule at its surface. In some embodiments, the isolated T cell of the invention expresses a CAR and a viral protein that downregulates MHC Class I cell surface expression.

In some embodiments, the viral protein does not significantly decrease cell surface expression of the CAR relative to cell surface expression level of the CAR of an isolated T cell that comprises the CAR but does not comprise the viral protein.

In some embodiments, the isolated T cell can further comprise an NK cell antagonist. In some embodiments, the NK cell antagonist can be an anti-NK cell inhibitory receptor antibody. In some embodiments, the anti-NK cell inhibitory receptor antibody comprises a single-chain variable fragment (scFv). In some embodiments, the anti-NK cell inhibitory receptor antibody specifically binds to a killer cell immunoglobulin-like receptor (KIR), a CD94-NKG2A/C/E heterodimer, 2B4 (CD244) receptor, or Killer cell lectin-like receptor G1 (KLRG1) receptor. In some embodiments, the KIR can be KIR2DL1, KIR2DL2, KIR2DL3, KIR3DL1, KIR3DL2, KIR3DL3, KIR2DL5A, KIR2DL5B, or KIR2DL4.

In some embodiments, the isolated T cell exhibits improved in vivo persistence relative to in vivo persistence of an isolated T cell that comprises the CAR but does not comprise the viral protein.

In some embodiments, the isolated T cell elicits no or a reduced graft-versus-host disease (GVHD) response in a histoincompatible recipient as compared to the GVHD response elicited by an isolated T cell that comprises the CAR but does not comprise the viral protein. In some embodiments, the recipient is a human or a monkey.

In another aspect, the invention provides a CAR-T cell population comprising: a plurality of isolated T cells comprising a viral protein which decreases cell surface expression level of major histocompatibility complex (MHC) Class I relative to cell surface expression level of MHC Class I of an isolated T cell that does not comprise the viral protein, and a chimeric antigen receptor (CAR) comprising an extracellular ligand-binding domain, a transmembrane domain, and an intracellular signaling domain.

In some embodiments, cell surface expression level of MHC Class I is decreased by at least about 50%, 55%, 60%, 65%, 70%, 75%, 80%, 85%, 90%, 95%, 99%, or 100% relative to cell surface expression level of MHC Class I on T cells that do not comprise the viral protein. In some embodiments, cell surface expression levels of MHC Class I may be measured by flow cytometry.

In some embodiments, administering a T-cell of the invention comprising a CAR and a viral protein selected reduces rejection by at least 50%, 60%, 70%, 80%, 90%, 95%, 99%, or 100% as compared to administering a T-cell not expressing a viral protein. In some embodiments, the viral protein is selected from Table 1.

In some embodiments, administering a T-cell of the invention comprising a CAR and a viral protein increases the duration of the response by at least 50%, 60%, 70%, 80%, 90%, 95%, 99%, or 100% as compared to administering a T-cell not expressing a viral protein. In some embodiments, the viral protein is selected from Table 1.

In some embodiments, administering a T-cell of the invention comprising a CAR and a viral protein improves persistence by at least 50%, 60%, 70%, 80%, 90%, 95%, 99%, or 100% as compared to administering a T-cell not expressing a viral protein. In some embodiments, the viral protein is selected from Table 1.

In some embodiments, administering a T-cell of the invention comprising a CAR and a viral protein reduces the incidence of GVHD by at least 50%, 60%, 70%, 80%, 90%, 95%, 99%, or 100% as compared to administering a T-cell not expressing viral protein. In some embodiments, the viral protein is selected from Table 1.

In another aspect, the invention provides a method of generating an isolated T cell, wherein the method comprises modifying a T-cell expressing CAR to express a viral protein, wherein the CAR comprises an extracellular ligand-binding domain, a transmembrane domain, and an intracellular signaling domain. In some embodiments, the method can further comprise a step of modifying the T cell to express an anti-NK cell antagonist.

In some embodiments, a polynucleotide that encodes viral protein can be introduced to the cell by, for example without limitation, electroporation.

In some embodiments, a polynucleotide that encodes the chimeric antigen receptor can be introduced to the cell by a transposon/transposase system, a viral-based gene transfer system or electroporation.

In some embodiments, the viral-based gene transfer system comprises recombinant retrovirus or lentivirus.

In some embodiments, the step of modifying the T cell to express an anti-NK cell antagonist comprises introducing a polynucleotide that encodes the NK cell antagonist to the cell by, for example without limitation, electroporation.

In another aspect, the invention provides pharmaceutical composition for use in treating a disorder, wherein the composition comprises an isolated T cell comprising a viral protein which decreases cell surface expression level of major histocompatibility complex (MHC) Class I relative to cell surface expression level of MHC Class I of an isolated T cell that does not comprise the viral protein, and a chimeric antigen receptor (CAR) comprising an extracellular ligand-binding domain, a transmembrane domain, and an intracellular signaling domain. In some embodiment, the composition further comprises an NK cell antagonist.

In some embodiments, the disorder can be cancer, autoimmune disease, or infection. In some embodiments, the cells can be provided more than once. In some embodiments, the cells can be provided to the subject at least about 1, 2, 3, 4, 5, 6, 7, or more days apart. In some embodiments, the disorder can be a viral disease, a bacterial disease, a cancer, an inflammatory disease, an immune disease, or an aging-associated disease.

In another aspect, the invention provides a method for treating a disorder in a subject, wherein the method comprises administering an isolated T cell comprising a viral protein which decreases cell surface expression level of major histocompatibility complex (MHC) Class I relative to cell surface expression level of MHC Class I of an isolated T cell that does not comprise the viral protein, and a chimeric antigen receptor (CAR) comprising an extracellular ligand-binding domain, a transmembrane domain, and an intracellular signaling domain. In some embodiments, the method further comprises administering an NK cell antagonist. In another aspect, the invention provides a method of reducing GVHD in a recipient subject comprising administering to said subject a population of T-cells expressing a CAR and a viral protein. In another aspect, the invention provides a method of improving persistence in a recipient subject comprising administering to said subject a population of T-cells expressing a CAR and a viral protein. In another aspect, the invention provides a method of lengthening the time of a durable response in a recipient subject comprising administering to said subject a population of T-cells expressing a CAR and a viral protein. In some embodiments, the viral protein is selected from Table 1.

In some embodiments of the method, the isolated T cell can further comprise an NK cell antagonist.

In some embodiments of the method, the NK cell antagonist can be an anti-NK cell inhibitor receptor antibody. In some embodiments, the anti-NK cell inhibitory receptor antibody can be an anti-KIR antibody.

In some embodiments, the cells can be provided to the subject more than once. In some embodiments of the method, the subject can be previously treated with a therapeutic agent prior to administration of the isolated T cell. In some embodiments, the therapeutic agent can be an antibody or chemotherapeutic agent. In some embodiments, the disorder can be a viral disease, a bacterial disease, a cancer, an inflammatory disease, an immune disease, or an aging-associated disease.

In some embodiments, the cancer can be a hematological malignancy or a solid cancer. In some embodiments, the hematological malignancy can be selected from acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), or multiple myeloma (MM). In some embodiments, the solid cancer can be selected from biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer.

In some embodiments, the method can comprises administering to the subject one or more additional therapeutic agent(s). In some embodiments, the additional therapeutic agent can be an antibody or chemotherapeutic agent.

In another aspect, the invention provides a polynucleotide encoding (i) a viral protein which decreases cell surface expression level of a major histocompatibility complex (MHC) Class I molecule relative to cell surface expression level of MHC Class I molecule of an isolated T cell that does not comprise the viral protein and (ii) a chimeric antigen receptor (CAR), wherein (i) are (ii) co-expressed. In some embodiments, the coding sequence for (i) and (ii) are operably linked to the same promoter. In some embodiments, the polynucleotide further encodes (iii) an NK cell antagonist.

In another aspect, the invention provides a vector comprising a polynucleotide encoding (i) a viral protein which decreases cell surface expression level of a major histocompatibility complex (MHC) Class I molecule relative to cell surface expression level of MHC Class I molecule of an isolated T cell that does not comprise the viral protein and (ii) a chimeric antigen receptor (CAR), wherein (i) are (ii) co-expressed. In some embodiments, the vector can be a viral vector.

### BRIEF DESCRIPTION OF THE DRAWINGS

FIG. 1A and FIG. 1B depict graphs summarizing the results of cytometry analysis of CAR+ Jurkat cells that co-express viral protein US11 (FIG. 1A) or K5 (FIG. 1B). Y-axis indicates CAR expression, and X-axis indicates MHC Class I expression.

FIG. 2 depicts a graph summarizing the MHC Class I surface expression levels of cells that do not express either CAR or viral protein (left columns, “−”) compared to cells expressing CAR and the indicated viral protein (right columns, “+”). GMFI=geometric mean fluorescence intensity.

### DETAILED DESCRIPTION

The present invention provides methods and compositions for improving in vivo persistence and therapeutic efficacy of CAR-T cells. Compositions and methods that downregulate major histocompatibility class (MHC) I cell surface expression are provided herein. Also provided are uses of such compositions and methods for improving the functional activities of isolated T cells, such as CAR-T cells. Also provided herein are CAR-T cells having improved persistence, and methods of using such CAR-T cells for treating a disorder.

### General Techniques

The practice of the invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

As used herein “autologous” means that cells, a cell line, or population of cells used for treating subjects are originating from said subject.

As used herein “allogeneic” means that cells or population of cells used for treating subjects are not originating from said subject but from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, B cells, natural killer (NK) cells, natural killer T (NKT) cells, mast cells, and myeloic-derived phagocytes.

As used herein, the term “expression” refers to the transcription and/or translation of a particular nucleotide sequence driven by a promoter.

As used herein, “expression vector” refers to a vector comprising a recombinant polynucleotide comprising expression control sequences operatively linked to a nucleotide sequence to be expressed. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

“Promoter” and “promoter sequence” are used interchangeably and refer to a DNA sequence capable of controlling the expression of a coding sequence or functional RNA. In general, a coding sequence is located 3′ to a promoter sequence. It is understood by those skilled in the art that different promoters may direct the expression of a gene in different tissues or cell types, or at different stages of development, or in response to different environmental or physiological conditions.

In any of the vectors of the present invention, the vector optionally comprises a promoter disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “extracellular ligand-binding domain” as used herein refers to an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state.

The term “stalk domain” is used herein to refer to any oligo- or polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on a T cell that specifically binds with a co-stimulatory ligand, thereby mediating a co-w stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin β receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)₂, and Fv), and any other modified configuration of the immunoglobulin molecule that comprises an antigen recognition site including, for example without limitation, single chain (scFv) and domain antibodies (including, for example, shark and camelid antibodies), and fusion proteins comprising an antibody. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen-binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “specifically binds” to a target is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. It is also understood that by reading this definition, for example, an antibody (or moiety or epitope) that specifically binds to a first target may or may not specifically bind to a second target. As such, “specific binding” does not necessarily require (although it can include) exclusive binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of subjects having a disease (e.g., cancer).

“Ameliorating” means a lessening or improvement of one or more symptoms as compared to not administering a treatment. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, a “subject” is any mammal, e.g a human, or a monkey. Mammals include, but are not limited to, farm animals, sport animals, pets, primates, horses, dogs, cats, mice and rats. In an exemplary embodiment, the subject is a human. In an exemplary embodiment, the subject is a monkey, e.g. a cynomolgus monkey.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

As used herein, “alloreactivity” refers to the ability of T cells to recognize MHC complexes that were not encountered during thymic development. Alloreactivity manifests itself clinically as hostversus graft rejection and graft versus host disease.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the invention encompasses not only the entire group listed as a whole, but each member of the group individually and all possible subgroups of the main group, but also the main group absent one or more of the group members. The invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the invention. The materials, methods, and examples are illustrative only and not intended to be limiting.

### Improved Isolated T Cells

Compositions and methods that downregulate major histocompatibility class (MHC) I cell surface expression are provide herein. Also provided are uses of such compositions and methods for improving the functional activities of isolated T cells, such as CAR-T cells. The methods and compositions provided herein are useful for improving in vivo persistence and therapeutic efficacy of CAR-T cells.

Isolated T cells provided herein express: (i) a viral protein which downregulates MHC Class I cell surface expression and (ii) a chimeric antigen receptor (CAR). Advantageously, the isolated T cells provided herein exhibit improved in vivo persistence relative to cells that does not express the viral protein. Preferably, the viral protein does not decrease CAR cell surface expression of the isolated T cell.

In some embodiments, an isolated T cell provided herein further comprises (iii) a protein that inhibits NK cell activity. For example, the isolated T cells can express an NK cell antagonist, including for example an anti-NK cell inhibitory receptor antibody. In some embodiments, the anti-NK cell inhibitory receptor antibody specifically binds to a killer cell immunoglobulin-like receptor (KIR), a CD94-NKG2A/C/E heterodimer, 2B4 (CD244) receptor, a Killer cell lectin-like receptor G1 (KLRG1) receptor, {Tom: Please list any other possibilities}. The KIR can be, for example without limitation, KIR2DL1, KIR2DL2, KIR2DL3, KIR3DL1, KIR3DL2, KIR3DL3, KIR2DL5A, KIR2DL5B, and KIR2DL4. Anti-NK cell inhibitory receptor antibodies useful in the invention preferably (a) target a receptor which generates a strong inhibitory signal, (b) are expressed primarily in NK cells, and/or (c) target a specific and conserved epitope so it is applicable to patients with a wide range of allelic variability.

The viral protein can be any viral protein that can interfere with interfere with cell surface expression of an MHC class I molecule. Exemplary viral proteins useful in the present invention include without limitation BFP, ICP47, K3, K5, E19, U3, US6, US2, US11, Nef, U21, EBNA1, UL49.5, BNLF2a, CPXV203, and US10. In some embodiments, the viral protein can be a cytomegalovirus (CMV) protein, adenovirus protein, herpesvirus protein, or human immunodeficiency virus protein. To determine if a viral protein downregulates cell surface expression of an MHC class I molecule, surface expression levels of MHC class I can be assayed in cells expressing the viral protein and relative to levels on cells that do not express the viral protein. Assays for determining surface expression levels of MHC class I are known in the art. For example, cells can be stained for surface MHC Class I expression with an antibody against HLA-A, B, C for subsequent flow cytometry (FACS) analysis.

In some embodiments, cell surface expression levels of MHC Class I on T cells expressing a viral protein may be decreased by at least about 30%, 35%, 40%, 45%, 50%, 55%, 60%, 65%, 70%, 75%, 80%, 85%, 90%, 95%, 99%, or 100% relative to cell surface expression levels of MHC Class I on T cells that do not comprise the viral protein.

In some embodiments, an isolated T cell of the invention comprises (e.g. expresses) a viral protein sequence as listed in Table 1, or a viral sequence with viral sequence.

TABLE 1
Exemplary Viral Protein Sequences

| | | SEQ |
|---|---|---|
| Viral | | ID. |
| protein | Sequence | NO: |
| ICP47 | MSWALEMADTFLDTMRVGPRTYADVRDEINKRGRED | 1 |
| | REAARTAVHDPERPLLRSPGLLPEIAPNASLGVAHRR | |
| | TGGTVTDSPRNPVTR | |
| K3 | MEDEDVPVCWICNEELGNERFRACGCTGELENVHRS | 2 |
| | CLSTWLTISRNTACQICGVVYNTRVVWRPLREMTLLP | |
| | RLTYQEGLELIVFIFIMTLGAAGLAAATWVWLYIVGGH | |
| | DPEIDHVAAAAYYVFFVFYQLFVVFGLGAFFHMMRHV | |
| | GRAYAAVNTRVEVFPYRPRPTSPECAVEEIELQEILPR | |
| | GDNQDEEGPAGAAPGDQNGPAGAAPGDQDGPADG | |
| | APVHRDSEESVDEAAGYKEAGEPTHNDGRDDNVEPT | |
| | AVGCDCNNLGAERYRATYCGGYVGAQSGDGAYSVS | |
| | CHNKAGPSSLVDILPQGLPGGGYGSMGVIRKRSAVSS | |
| | ALMFH | |
| K5 | MASKDVEEGVEGPICWICREEVGNEGIHPCACTGELD | 3 |
| | VVHPQCLSTWLTVSRNTACQMCRVIYRTRTQWRSRL | |
| | NLWPEMERQEIFELFLLMSVVVAGLVGVALCTWTLLVI | |
| | LTAPAGTFSPGAVLGFLCFFGFYQIFIVFAFGGICRVS | |
| | GTVRALYAANNTRVTVLPYRRPRRPTANEDNIELTVLV | |
| | GPAGGTDEEPTDESSEGDVASGDKERDGSSGDEPDG | |
| | GPNDRAGLRGTARTDLCAPTKKPVRKNHPKNNG | |
| E19 | MIRYIILGLLTLASAHGTTQKVDFKEPACNVTFAAEAN | 4 |
| | ECTTLIKCTTEHEKLLIRHKNKIGKYAVYAIWQPGDTT | |
| | EYNVTVFQGKSHKTFMYTFPFYEMCDITMYMSKQYKLW | |
| | PPQNCVENTGTFCCTAMLITVLALVCTLLYIKYKSRRS | |
| | FIEEKKMP | |
| US3 | MKPVLVLAILAVLFLRLADSVPRPLDVVVSEIRSAHFR | 5 |
| | VEENQCWFHMGMLYFKGRMSGNFTEKHFVNVGIVSQ | |
| | SYMDRLQVSGEQYHHDERGAYFEWNIGGHPVTHTVD | |
| | MVDITLSTRWGDPKKYAACVPQVRMDYSSQTINWYL | |
| | QRSMRDDNWGLLFRTLLVYLFSLVVLVLLTVGVSARL | |
| | RFI | |
| US6 | MDLLIRLGFLLMCALPTPGERSSRDPKTLLSLSPRQQA | 6 |
| | CVPRTKSHRPVCYNDTGDCTDADDSWKQLGEDFAH | |
| | QCLQAAKKRPKTHKSRPNDRNLEGRLTCQRVRRLLP | |
| | CDLDIHPSHRLLTLMNNCVCDGAVWNAFRLIERHGFF | |
| | AVTLYLCCGITLLVVILALLCSITYESTGRGIRR | |
| US2 | MNNLWKAWVGLWTSMGPLIRLPDGITKAGEDALRPW | 7 |
| | KSTAKHPWFQIEDNRCYIDNGKLFARGSIVGNMSRFV | |
| | FDPKADYGGVGENLYVHADDVEFVPGESLKWNVRNL | |
| | DVMPIFETLALRLVLQGDVIWLRCVPELRVDYTSSAYM | |
| | WNMQYGMVRKSYTHVAWTIVFYSINITLLVLFIVYVTV | |
| | DCNLSMMWMRFFVC | |
| US11 | MNLVMLILALWAPVAGSMPELSLTLFDEPPPLVETEPL | 8 |
| | PPLSDVSEYRVEYSEARCVLRSGGRLEALWTLRGNLS | |
| | VPTPTPRVYYQTLEGYADRVPTPVEDVSESLVAKRYW | |
| | LRDYRVPQRTKLVLFYFSPCHQCQTYYVECEPRCLVP | |
| | WVPLWSSLEDIERLLFEDRRLMAYYALTIKSAQYTLM | |
| | MVAVIQVFWGLYVKGWLHRHFPWMFSDQW | |
| Nef | MGGKWSKSSVIGWPTVRERMRRAEPAADRVGAASR | 9 |
| | DLEKHGAITSSNTAATNAACAWLEAQEEEEVGFPVTP | |
| | QVPLRPMTYKAAVDLSHFLKEKGGLEGLIHSQRRQDIL | |
| | DLWIYHTQGYFPDWQNYTPGPGVRYPLTFGWCYKLV | |
| | PVEPDKIEEANKGENTSLLHPVSLHGMDDPEREVLEW | |
| | RFDSRLAFHHVARELHPEYFKNC | |
| U21 | MWTILLFCVPVIYGELYPDFCPLAVVDFDVNATVDDLL | 10 |
| | LFDISLSKQCSDDKIRHSAVAAMTDNAFFFGNSETQIE | |
| | TDFGKYLAFNCYQVFSTLNHFLFKNFKKTKGLMKRYD | |
| | KLCLDVESYIHIQIICSPFKSFIRLRRMNETGISPRIL | |
| | ETTFYLQNKRNSTWVAIKNYLGEDDPFTYRIWHTLTHA | |
| | KNFLINSCENDFNQLFFWQRKYLSLAKTFEATFKQGFN | |
| | PMIEQRNEQRYRTNNIDCSFSKFRQNGVKVAVCKYTG | |
| | WGVSGFGSLEVLQKIKSPFGEEWKRVGFNSTGAFTP | |
| | LYGSDVLWGLIFLRVEMTTYVCTCTNKNTGTQIQVTLP | |
| | DVDLDLLDSEKTSSNVFVDMLCYTLIAILFLAFVTAVV | |
| | LLGVSCLDGVQKVLTWPLQHIQKEPVSEKIINLTNLMF | |
| | GQEPLPKKESLKQQCL | |
| US10 | MLRRGSLRNPLAICLLVWWLGVVAAATEETREPTYFTC | 11 |
| | GCVIQNHVLKGAVKLYGQFPSPKTLRASAWLHDGEN | |
| | HERHRQPILVEGTATATEALYILLPTELSSPEGNRPRN | |
| | YSATLTLASRDCYERFVCPVYDSGTPMGVLMNLTYL | |
| | WYLGDYGAILKIYFGLFCGACVITRSLLLICGYYPPRE | |
| EBNA-1 | MSDEGPGTGPGNGLGEKGDTSGPEGSGGSGPQRR | 12 |
| | GGDNHGRGRGRGRGRGGGRPGAPGGSGSGPRHR | |
| | DGVRRPQKRPSCIGCKGTHGGTGAGAGAGGAGAGG | |
| | AGAGGGAGAGGGAGGAGGAGGAGAGGGAGAGGG | |
| | AGGAGGAGAGGGAGAGGGAGGAGAGGGAGGAGG | |
| | AGAGGGAGAGGGAGGAGAGGGAGGAGGAGAGGG | |
| | AGAGGAGGAGGAGAGGAGAGGGAGGAGGAGAGGA | |
| | GAGGAGAGGAGAGGAGGAGAGGAGGAGAGGAGGA | |
| | GAGGGAGGAGAGGGAGGAGAGGAGGAGAGGAGG | |
| | AGAGGAGGAGAGGGAGAGGAGAGGGGRGRGGSG | |
| | GRGRGGSGGRGRGGSGGRRGRGRERARGGSRERA | |
| | RGRGRGRGEKRPRSPSSQSSSSGSPPRRPPPGRRP | |
| | FFHPVGEADYFEYHQEGGPDGEPDVPPGAIEQGPAD | |
| | DPGEGPSTGPRGQGDGGRRKKGGWFGKHRGQGGS | |
| | NPKFENIAEGLRALLARSHVERTTDEGTWVAGVFVYG | |
| | GSKTSLYNLRRGTALAIPQCRLTPLSRLPFGMAPGPG | |
| | PQPGPLRESIVCYFMVFLQTHIFAEVLKDAIKDLVMTK | |
| | PAPTCNIRVTVCSFDDGVDLPPWFPPMVEGAAAEGD | |
| | DGDDGDEGGDGDEGEEGQE | |
| BNLF2a | MVHVLERALLEQQSSACGLPGSSTETRPSH | 13 |
| | PCPEDPDVSRLRLLLVVLCVLFGLLCLLLI | |
| UL49.5 | MGSITASFILITMQILFFCEDSSGEPNFAERNFWHASC | 14 |
| | SARGVYIDGSMITTLFFYASLLGVCVALISLAYHACFR | |
| | LFTRSVLRSTR | |
| CPXV203 | MRSLVIVLLFPSIIYSMVIRRCEKMEEETWKLKIGMCI | 15 |
| | QAKDFYSKRTDCSVHRPDVGGGLITEGNGYRVVVHDQ | |
| | CEEPNPFIIATTKQTHFGVTHSYIEFSNSNTGAPENIP | |
| | DCSKHILISVYCDQEASGLDFHTLKYVESNYLHITVKY | |
| | DTSCINHLGVNYSFMNECERKLTSIYETDTLTCGAKDI | |
| | QTRDKYLKTCTNTKFDRSVYKTHMQKSKILHVKTEL | |

In some embodiments, an isolated T cell of the invention comprises (e.g. expresses) ICP47. In some embodiments, an isolated T cell of the invention comprises (e.g. expresses) K3, e.g. In some embodiments, an isolated T cell of the invention comprises (e.g. expresses) K5. In some embodiments, an isolated T cell of the invention comprises (e.g. expresses) E19. In some embodiments, an isolated T cell of the invention comprises (e.g. expresses) US3. In some embodiments, an isolated T cell of the invention comprises (e.g. expresses) US6. In some embodiments, an isolated T cell of the invention comprises (e.g. expresses) US2. In some embodiments, an isolated T cell of the invention comprises (e.g. expresses) US11. In some embodiments, an isolated T cell of the invention comprises (e.g. expresses) Nef. In some embodiments, an isolated T cell of the invention comprises (e.g. expresses) U21. In some embodiments, an isolated T cell of the invention comprises (e.g. expresses) US10. In some embodiments, an isolated T cell of the invention comprises (e.g. expresses) EBNA-1. In some embodiments, an isolated T cell of the invention comprises (e.g. expresses) BNLF2a. In some embodiments, an isolated T cell of the invention comprises (e.g. expresses) UL49.5. In some embodiments, an isolated T cell of the invention comprises (e.g. expresses) CPXV203.

The invention encompasses modifications to the proteins of the invention embodiments shown in Table 1, including functionally equivalent proteins having modifications which do not significantly affect their properties and variants which have enhanced or decreased activity and/or affinity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag.

Substitution variants have at least one amino acid residue in the viral protein removed and a different residue inserted in its place. Conservative substitutions are shown in Table 2 under the heading of “conservative substitutions.” If such substitutions result in a change in biological activity, then more substantial changes, denominated “exemplary substitutions” in Table 2, or as further described below in reference to amino acid classes, may be introduced and the products screened.

TABLE 2
Amino Acid Substitutions

| Original Residue | | |
|---|---|---|
| (naturally occurring | Conservative | Exemplary |
| amino acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Viral proteins may be synthesized in situ in the cell after introduction of polynucleotides encoding the viral proteins into the cell. Alternatively, viral proteins may be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g. retroviruses, adenoviruses), liposomes, and the like. Transient transformation methods include, for example without limitation, microinjection, electroporation or particle bombardment. Polynucleotides may be included in vectors, such as for example plasmid vectors or viral vectors.

In some embodiments, an isolated T cell of the invention can comprise at least one viral protein and at least one CAR. In some embodiments, an isolated T cell can comprise at least a population of different viral proteins and at least one CAR. In some embodiments, an isolated T cell can comprise at least one viral protein and a population of CARs, each CAR comprising different extracellular ligand-binding domains.

In some embodiments of an isolated T cell provided herein, a CAR can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain specifically binds to a target of interest. The target of interest can be any molecule of interest, including, for example without limitation BCMA, EGFRvIII, Flt-3, WT-1, CD20, CD23, CD30, CD38, CD70, CD33, CD133, MHC-WT1, TSPAN10, MHC-PRAME, Liv1, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17 (Mucin17, Muc3, Muc3), FAP alpha (Fibroblast Activation Protein alpha), Ly6G6D (Lymphocyte antigen 6 complex locus protein G6d, c6orf23, G6D, MEGT1, NG25), RNF43 (E3 ubiquitin-protein ligase RNF43, RING finger protein 43).

In some embodiments, the extracellular ligand-binding domain comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID NO: 16), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the invention is responsible for intracellular signaling following the binding of extracellular ligand-binding domain to the target resulting in the activation of the immune cell and immune response. The intracellular signaling domain has the ability to activate of at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3 signaling domain. In some embodiments the intracellular signaling domain of the CAR of the invention comprises a domain of a co-stimulatory molecule.

In some embodiments, the intracellular signaling domain of a CAR of the invention comprises a part of co-stimulatory molecule selected from the group consisting of fragment of 41BB (GenBank: AAA53133.) and CD28 (NP_006130.1).

CARs are expressed on the surface membrane of the cell. Thus, the CAR can comprise a transmembrane domain. Suitable transmembrane domains for a CAR disclosed herein have the ability to (a) be expressed at the surface of a cell, preferably an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, said transmembrane comprises a part of human CD8α chain. In some embodiments, CARs disclosed herein can comprise an extracellular ligand-binding domain that specifically binds BCMA, CD8α human stalk and transmembrane domains, the CD3 signaling domain, and 4-1BB signaling domain. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides may be synthesized in situ in the cell after introduction of polynucleotides encoding the CAR polypeptides into the cell. Alternatively, CAR polypeptides may be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g. retroviruses, adenoviruses), liposomes, and the like. Transient transformation methods include, for example without limitation, microinjection, electroporation or particle bombardment. Polynucleotides may be included in vectors, such as for example plasmid vectors or viral vectors.

Also provided herein are isolated T cells obtained according to any one of the methods described herein. Any immune cell capable of expressing heterologous DNAs can be used for the purpose of expressing the viral protein and CAR of interest. In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+T-lymphocytes and CD8+T-lymphocytes.

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, may be used. In some embodiments, cells can be derived from a healthy donor, from a subject diagnosed with cancer or from a subject diagnosed with an infection. In some embodiments, cells can be part of a mixed population of cells which present different phenotypic characteristics.

Also provided herein are cell lines obtained from a transformed T cell according to any of the methods described herein. In some embodiments, an isolated T cell according to the invention comprises a polynucleotide encoding a viral protein. In some embodiments, an isolated T cell according to the invention comprises a polynucleotide encoding a viral protein and a polynucleotide encoding a CAR. In some embodiments, an isolated T cell according to the invention comprises a polynucleotide encoding a viral protein, a polynucleotide encoding a CAR, and a polynucleotide encoding an NK cell antagonist.

The isolated T cells of the invention can be activated and expanded, either prior to or after genetic modification of the T cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. Generally, the T cells of the invention can be expanded, for example, by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, TGFp, and TNF, or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics

In some embodiments, the cells of the invention can be expanded by co-culturing with tissue or cells. The cells can also be expanded in vivo, for example in the subject's blood after administrating the cell into the subject.

In another aspect, the invention provides compositions (such as a pharmaceutical compositions) comprising any of the cells of the invention. In some embodiments, the composition comprises an isolated T cell comprising a polynucleotide encoding any of the viral proteins described herein, and a polynucleotide encoding a CAR. In some embodiments, the cell further comprises a polynucleotide encoding an NK cell antagonist. In some embodiments, the NK cell antagonist is an anti-NK cell inhibitory receptor antibody.

Expression vectors and administration of polynucleotide compositions are further described herein.

In another aspect, the invention provides a method of making any of the polynucleotides described herein.

Polynucleotides complementary to any such sequences are also encompassed by the invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the invention. Further, alleles of the genes comprising the polynucleotide sequences provided herein are within the scope of the invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding a viral protein or a CAR disclosed herein may exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an imRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro sequence or pre sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. In some embodiments, nucleic acid sequences of the invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Methods of preparing immune cells for use in immunotherapy are provided herein. In some embodiments, the methods comprise introducing a viral protein and a CAR into immune cells, and expanding the cells. In some embodiments, the invention relates to a method of engineering an immune cell comprising: providing a cell and expressing a viral protein which downregulates MHC cell surface expression, and expressing at the surface of the cell at least one CAR. In some embodiments, the method comprises: transfecting the cell with at least one polynucleotide encoding a viral protein, and at least one polynucleotide encoding a CAR, and expressing the polynucleotides in the cell. In some embodiments, the method comprises: transfecting the cell with at least one polynucleotide encoding a viral protein, at least one polynucleotide encoding a CAR, and at least one polynucleotide encoding an NK cell antagonist, and expressing the polynucleotides in the cell.

In some embodiments, the polynucleotides encoding the viral protein and CAR are present in one or more expression vectors for stable expression in the cells. In some embodiments, the polynucleotides are present in viral vectors for stable expression in the cells. In some embodiments, the viral vectors may be for example, lentiviral vectors or adenoviral vectors.

In some embodiments, polynucleotides encoding polypeptides according to the present invention can be mRNA which is introduced directly into the cells, for example by electroporation. In some embodiments, cytoPulse technology can be used to transiently permeabilize living cells for delivery of material into the cells. Parameters can be modified in order to determine conditions for high transfection efficiency with minimal mortality.

Also provided herein are methods of transfecting a T cell. In some embodiments, the method comprises: contacting a T cell with RNA and applying to T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting T cell comprising contacting said T cell with RNA and applying to T cell an agile pulse sequence comprising: (a) an electrical pulse with a voltage of about 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250, of 2250, 2300, 2350, 2400, 2450, 2500, 2550, 2400, 2450, 2500, 2600, 2700, 2800, 2900 or 3000V with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, the method can further comprise a step of genetically modifying a cell by inactivating at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, CD52, GR, deoxycytidine kinase (DCK), PD-1, and CTLA-4. In some embodiments the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or Cas9 endonuclease.

In another aspect, a step of genetically modifying cells can comprise: modifying T cells by inactivating at least one gene expressing a target for an immunosuppressive agent, and; expanding the cells, optionally in presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others may act through activation of T cells or by inhibiting the activation of helper cells. The methods according to the invention allow conferring immunosuppressive resistance to T cells for immunotherapy by inactivating the target of the immunosuppressive agent in T cells. As non-limiting examples, targets for immunosuppressive agent can be a receptor for an immunosuppressive agent such as for example without limitation CD52, glucocorticoid receptor (GR), FKBP family gene members, and cyclophilin family gene members.

### Therapeutic Methods

Isolated T cells obtained by the methods described above, or cell lines derived from such isolated T cells, can be used as a medicament. In some embodiments, such a medicament can be used for treating a disorder such as for example a viral disease, a bacterial disease, a cancer, an inflammatory disease, an immune disease, or an aging-associated disease. In some embodiments, the cancer can be selected from the group consisting of gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, an isolated T cells according to the invention, or cell line derived from the isolated T cells cells, can be used in the manufacture of a medicament for treatment of a disorder in a subject in need thereof. In some embodiments, the disorder can be, for example, a cancer, an autoimmune disorder, or an infection.

Also provided herein are methods for treating subjects. In some embodiments the method comprises providing an isolated T cell of the invention to a subject in need thereof. In some embodiments, the method comprises a step of administering isolated T cells of the invention to a subject in need thereof.

In some embodiments, isolated T cells of the invention can undergo robust in vivo T cell expansion and can persist for an extended amount of time.

Methods of treatment of the invention can be ameliorating, curative or prophylactic. The method of the invention may be either part of an autologous immunotherapy or part of an allogenic immunotherapy treatment. The invention is particularly suitable for allogeneic immunotherapy. T cells from donors can be transformed into non-alloreactive cells using standard protocols and reproduced as needed, thereby producing CAR-T cells which may be administered to one or several subjects. Such CAR-T cell therapy can be made available as an “off the shelf” therapeutic product.

In another aspect, the invention provides a method of inhibiting tumor growth or progression in a subject who has a tumor, comprising administering to the subject an effective amount of isolated T cells as described herein. In another aspect, the invention provides a method of inhibiting or preventing metastasis of cancer cells in a subject, comprising administering to the subject in need thereof an effective amount of isolated T cells as described herein. In another aspect, the invention provides a method of inducing tumor regression in a subject who has a tumor, comprising administering to the subject an effective amount of isolated T cells as described herein.

In some embodiments, the isolated T cells herein can be administered parenterally in a subject. In some embodiments, the subject is a human.

In some embodiments, the method can further comprise administering an effective amount of a second therapeutic agent. In some embodiments, the second therapeutic agent is, for example, crizotinib, palbociclib, an anti-CTLA4 antibody, an anti-4-1BB antibody, a PD-1 antibody, or a PD-L1 antibody.

Also provided is the use of any of the isolated T cells provided herein in the manufacture of a medicament for the treatment of cancer or for inhibiting tumor growth or progression in a subject in need thereof.

In some embodiments, cell surface expression level of MHC Class I is decreased by at least about 50%, 55%, 60%, 65%, 70%, 75%, 80%, 85%, 90%, 95%, 99%, or 100% relative to cell surface expression level of MHC Class I on T cells that do not comprise the viral protein. In some embodiments, cell surface expression levels of MHC Class I may be measured by flow cytometry.

In some embodiments, administering a T-cell of the invention comprising a CAR and a viral protein selected from Table 1 reduces rejection by at least 50%, 60%, 70%, 80%, 90%, 95%, 99%, or 100% as compared to administering a T-cell not expressing a viral protein selected from Table 1.

In some embodiments, administering a T-cell of the invention comprising a CAR and a viral protein selected from Table 1 increases the duration of the response by at least 50%, 60%, 70%, 80%, 90%, 95%, 99%, or 100% as compared to administering a T-cell not expressing a viral protein selected from Table 1.

In some embodiments, administering a T-cell of the invention comprising a CAR and a viral protein selected from Table 1 improves persistence by at least 50%, 60%, 70%, 80%, 90%, 95%, 99%, or 100% as compared to administering a T-cell not expressing a viral protein selected from Table 1.

In some embodiments, administering a T-cell of the invention comprising a CAR and a viral protein selected from Table 1 reduces the incidence of GVHD by at least 50%, 60%, 70%, 80%, 90%, 95%, 99%, or 100% as compared to administering a T-cell not expressing viral protein selected from Table 1.

In some embodiments, the treatment can be in combination with one or more therapies against cancer selected from the group of antibodies therapy, chemotherapy, cytokines therapy, dendritic cell therapy, gene therapy, hormone therapy, laser light therapy and radiation therapy.

In some embodiments, treatment can be administrated into subjects undergoing an immunosuppressive treatment. Indeed, the invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T cells according to the invention within the subject. The administration of the cells or population of cells according to the invention may be carried out in any convenient manner, including by aerosol inhalation, injection, ingestion, transfusion, implantation or transplantation. The compositions described herein may be administered to a subject subcutaneously, intradermaliy, intratumorally, intranodally, intramedullary, intramuscularly, by intravenous or intralymphatic injection, or intraperitoneally. In one embodiment, the cell compositions of the invention are preferably administered by intravenous injection.

In some embodiments the administration of the cells or population of cells can comprise administration of, for example, about 10⁴ to about 10⁹ cells per kg body weight including all integer values of cell numbers within those ranges. In some embodiments the administration of the cells or population of cells can comprise administration of about 10⁵ to 10⁶ cells per kg body weight including all integer values of cell numbers within those ranges. The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

In some embodiments of the invention, cells are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, BCMA specific CAR-T cells are administered to a subject in conjunction with one or more of the following: an anti-PD-1 antibody (e.g., nivolumab, pembrolizumab, or PF-06801591), an anti-PD-L1 antibody (e.g., avelumab, atezolizumab, or durvalumab), an anti-OX40 antibody (e.g., PF-04518600), an anti-4-1BB antibody (e.g., PF-05082566), an anti-MCSF antibody (e.g., PD-0360324), an anti-GITR antibody, and/or an anti-TIGIT antibody. In further embodiments, the isolated T cells cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the cell compositions of the invention are administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In some embodiments, the cell compositions of the invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the invention. In some embodiments, expanded cells are administered before or following surgery.

### Kits

The invention also provides kits for use in the instant methods. Kits of the invention include one or more containers comprising an isolated T cell comprising one or more polynucleotide(s) encoding a viral protein and a CAR as described herein, and instructions for use in accordance with any of the methods of the invention described herein. Generally, these instructions comprise a description of administration of the isolated T cell for the above described therapeutic treatments.

The instructions relating to the use of the isolated T cells as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an isolated T cell comprising a viral protein and a CAR. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the invention in any way. Indeed, various modifications of the invention in addition to those shown and described herein will become apparent to those skilled in the art from the foregoing description and fall within the scope of the appended claims.

### EXAMPLES

### Example 1: Downregulation of MHC Class I Molecule Cell Surface Expression on T Cells

This example illustrates the use of viral protein to downregulate cell surface expression of MHC Class I on isolated T cells expressing CAR.

In host versus graft (HvG) rejection, MHCs on donor cells are recognized by host T cells, which then eliminate the MHC-expressing donor cells. Thus, it is desirable to decrease cell surface expression of MHC Class I from allogeneic CAR-T cells to improve CAR-T cell persistence and/or ameliorate HvG rejection.

To determine the ability of various viral proteins to downregulate cell surface expression of MHC Class I on isolated T cells, Jurkat cells and primary human T cells were transduced with constructs encoding an anti-BCMA CAR, with or without co-expression of different CMV proteins, as shown in Table 3. BFP was used as a negative control protein. Only CAR+ cells are able to co-express CMV proteins based on the expression construct design.

TABLE 3

| Cell Type | CAR | Viral Protein |
|---|---|---|
| Jurkat | + | BFP |
| | − | BFP |
| | + | ICP47 |
| | + | K3 |
| | + | K5 |
| | + | E19 |
| | + | US3 |
| | + | US6 |
| | + | US2 |
| | + | US11 |
| Primary T cell | + | BFP |
| | − | BFP |
| | + | ICP47 |
| | + | K3 |
| | + | K5 |
| | + | E19 |
| | + | US3 |
| | + | US6 |
| | + | US2 |
| | + | US11 |

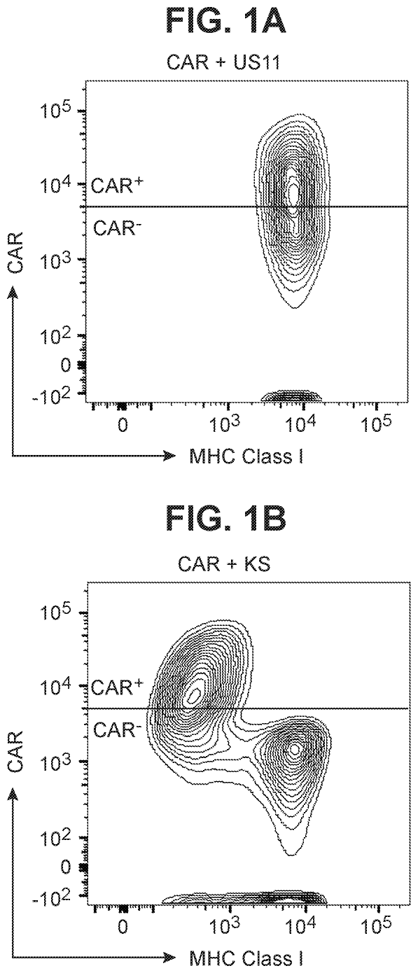

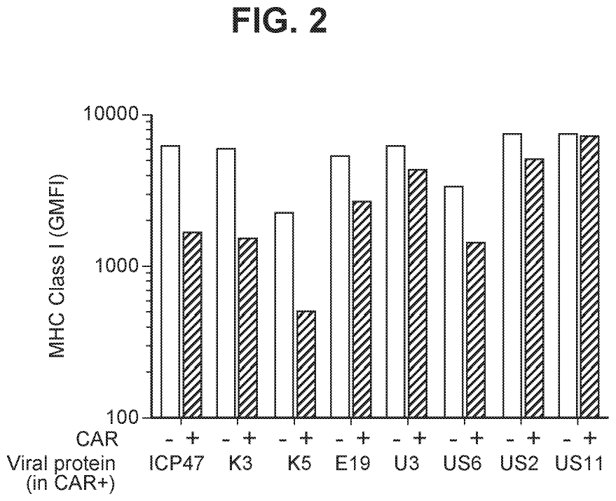

Functional MHC Class I complexes were detected on transduced cells using an antibody specific for HLA A/B/C, and anti-BCMA CAR surface expression was detected using biotinylated BCMA. Results are summarized in Tables 4 and 5, and FIGS. 1 and 2.

TABLE 4
Expression of MHC Class I on transduced Jurkat cells

| | CAR | MHC Class I | | |
|---|---|---|---|---|
| | expression | expression | | |
| Viral | (transduction | (MFI HLA | | |
| Protein | efficiency) | A/B/C) | SD | N |
| BFP | +(57.4%) | 10005 | 5747 | 62988 |
| | − | 7761 | 3749 | 45576 |
| ICP47 | +(57.1%) | 2064 | 1644 | 27873 |
| | − | 7325 | 3945 | 20504 |
| K3 | +(50.4%) | 2167 | 2285 | 24585 |
| | − | 7074 | 3975 | 23150 |
| K5 | +(40.7%) | 661 | 901 | 19859 |
| | − | 4683 | 4374 | 27338 |
| E19 | +(55.5%) | 3427 | 2789 | 27179 |
| | − | 6321 | 3666 | 20133 |
| US3 | +(54.8%) | 5316 | 3634 | 26575 |
| | − | 7181 | 3919 | 19988 |
| US6 | +(48.4%) | 1717 | 1258 | 23874 |
| | − | 5147 | 4394 | 23025 |
| US2 | +(43.3%) | 6078 | 3921 | 21313 |
| | − | 8126 | 4041 | 26380 |
| US11 | +(53.7%) | 8075 | 4457 | 26628 |
| | − | 8161 | 3982 | 20793 |

TABLE 5
Expression of MHC Class I on transduced primary T cells

| | CAR | MHC Class I | | |
|---|---|---|---|---|
| | expression | expression | | |
| Viral | (transduction | (MFI HLA | | |
| Protein | efficiency) | A/B/C) | SD | N |
| BFP | +(51.5%) | 14236 | 8852 | 17661 |
| | − | 11905 | 6822 | 16173 |
| ICP47 | +(64.2%) | 5936 | 7964 | 20594 |
| | − | 16695 | 9972 | 10996 |
| K3 | +(14.0%) | 10899 | 13433 | 1243 |
| | − | 12316 | 8438 | 7304 |
| K5 | +(55.2%) | 4478 | 6867 | 542 |
| | − | 15983 | 11982 | 404 |
| E19 | +(54.7%) | 14778 | 9020 | 21327 |
| | − | 12998 | 7507 | 17132 |
| US3 | +(38.1%) | 13022 | 7927 | 20993 |
| | − | 11081 | 6569 | 33147 |
| US6 | +(21.7%) | 9499 | 9271 | 8896 |
| | − | 14748 | 8072 | 31379 |
| US2 | +(22.8%) | 14474 | 8952 | 13988 |
| | − | 13216 | 7617 | 46639 |
| US11 | +(18.0%) | 14588 | 8610 | 14133 |
| | − | 13232 | 7272 | 63840 |

Varying levels of viral protein mediated MHC Class I downregulation was observed in CAR positive T cells. Viral protein mediated MHC Class I downregulation was observed in T cells expressing CAR and ICP47, K3, K5, E19, US3, US6, or US2 (Tables 4 and 5). For example, expression of K5 resulted in decreased cell surface expression of MHC Class I in CAR+ Jurkat cells accompanied by (FIG. 1, right; Table 4). No decrease in CAR expression was observed with this decrease in MHC Class I cell surface expression (FIG. 1). In contrast, expression of US11 did not decrease cell surface expression MHC Class I in CAR+ Jurkat cells (FIG. 1, left; Table 4). In some instances, MHC Class I down regulation was accompanied by lower CAR surface expression (data not shown).

Co-expression of each of the viral proteins ICP47, K3, K5, E19, U3, US6, and US2 with CAR resulted in decreased MHC Class I cell surface expression to various degrees (FIG. 2; Tables 4 and 5). In FIG. 2, the left column (−) represents cells that do not express either CAR or viral protein, and the right column (+) represents cells expressing CAR and the indicated viral protein. Only CAR+ cells are able to co-express CMV proteins based on the expression construct design.

These results demonstrate that viral proteins can reduce MHC Class I presentation on the surface of CAR-T cells.

### Example 2

This example illustrates the effect of co-expressing viral proteins on CAR-T cell activity and T cell-mediated alloreactivity both in vitro and in vivo. In this study, CAR-T cells co-expressing various CMV proteins are assessed using an in vitro T cell-mediated alloreactivity assay. MHC Class I cell surface expression is measured to determine the correlation between MHC Class I cell surface expression and alloreactivity.

To determine alloreactivity, a mixed lymphocyte reaction (MLR) assay is utilized. The assay involves incubating T cells from two allelic mismatched donors, then monitoring proliferation and cytokine release. In the assay, donors with mismatched MHC/TCR pairs respond by increasing proliferation and cytokine production compared to donors with matched MHC/TCR pairs.

The CAR-T cells are tested for target specific activity using in vitro cytotoxicity assays. These assays consist of mixing CAR-T cells with different ratios of target cells and measuring the extent of target cell killing using standard cytotoxicity measurements. CAR-T cells that show the largest reduction in alloreactivity while maintaining significant lytic activity in vitro are tested in vivo for activity and persistence using an NSG mouse models. Briefly, these CAR-T cells are administered to tumor-bearing mice and the growth of tumors compared to tumor growth in mice with unmodified T cells and mice with CAR-T cells that do not co-express viral proteins. Persistence of T cells is measured in peripheral blood, tumor and spleen. To simulate HvG reactions, the study includes the addition of T cells from an allelic mismatched donor to induce allorejection of the CAR-T cells.

### Example 3

This example illustrates the assessment of NK cell-mediated HvG of CAR-T cells that co-express viral proteins which downregulate MHC Class I cell surface expression.

Cells lacking allelic matched MHC Class I molecules are recognized as non-self by NK cells and eliminated (host versus graft rejection, or HvG). To determine the extent of NK cell-mediated HvG of CAR-T cells that co-express viral proteins which down regulate MHC Class I surface expression, in vitro and in vivo assays are used.

In vitro assays. NK cells are separately purified from allelic mismatched donors. The purified NK cells are assessed for their ability to induce HvG reactions using a MLR assay. The assay involves incubating T cells from two allelic mismatched donors, then monitoring proliferation and cytokine release. In the assay, donors with mismatched MHC/TCR pairs respond by increasing proliferation and cytokine production compared to donors with matched MHC/TCR pairs.

### Example 4

This example illustrates the use of anti-NK cell inhibitory receptor antibodies to attenuate NK cell-mediated elimination of CAR-T cells that express viral protein and have decreased MHC Class I cell surface expression.

Antibodies which specifically bind NK cell inhibitory receptors such as KIRs and lectin-like molecules are generated and tested for their ability to mimic MHC Class I inhibitory signaling. The antibodies assessed for their binding and mechanistic properties using biochemical assays and the same in vitro assays described above. Selected anti-NK cell inhibitory receptor antibodies are co-expressed as single chain antibodies (scFvs) on the surface of CAR-T cells co-expressing viral proteins and tested using the previously described in vitro assays. The CAR-T cells with decreased NK cell-mediated killing are evaluated for CAR-T cell activity, CAR-T cell persistence and HvG rejection in vivo using the previously described NSG mouse models.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

It is claimed:

1. An isolated T cell comprising
(i) a viral protein which decreases cell surface expression level of major histocompatibility complex (MHC) Class I relative to cell surface expression level of MHC Class I of an isolated T cell that does not comprise the viral protein, and
(ii) a chimeric antigen receptor (CAR) comprising an extracellular ligand-binding domain, a transmembrane domain, and an intracellular signaling domain.

2. The isolated T cell of claim 1, wherein the viral protein is a cytomegalovirus (CMV) protein, adenovirus protein, herpesvirus protein, or human immunodeficiency virus protein.

3. The isolated T cell of claim 1 or 2, wherein the viral protein is ICP47, K3, K5, E19, US3, US6, US2, U21, Nef, US10, or U21.

4. The isolated T cell of any one of claims 1 to 3, wherein the viral protein does not significantly decrease cell surface expression level of the CAR relative to cell surface expression level of the CAR of an isolated T cell that comprises the CAR but does not comprise the viral protein.

5. The isolated T cell of any one of claims 1 to 4, wherein the isolated T cell further comprises an NK cell antagonist.

6. The isolated T cell of claim 5, wherein the NK cell antagonist is an anti-NK cell inhibitory receptor agonist antibody or an anti-NK cell activating receptor antagonist antibody.

7. The isolated T cell of claim 6, wherein the anti-NK cell inhibitory receptor antibody comprises a single-chain variable fragment (scFv).

8. The isolated T cell of claim 6 or 7, wherein the anti-NK cell inhibitory receptor antibody specifically binds to a killer cell immunoglobulin-like receptor (KIR), a CD94-NKG2A/C/E heterodimer, a 2B4 (CD244) receptor, or a Killer cell lectin-like receptor G1 (KLRG1) receptor.

9. The isolated T cell of claim 8, wherein the KIR is KIR2DL1, KIR2DL2, KIR2DL3, KIR3DL1, KIR3DL2, KIR3DL3, KIR2DL5A, KIR2DL5B, or KIR2DL4.

10. The isolated T cell of any one of claims 1 to 9, wherein the isolated T cell exhibits improved in vivo persistence relative to in vivo persistence of a second isolated T cell, wherein the second isolated T cell comprises all components of the isolated T cell except it does not comprise the viral protein.

11. The isolated T cell of any one of claims 1 to 10, wherein the isolated T cell elicits no or a reduced graft-versus-host disease (GVHD) response in a histoincompatible recipient as compared to the GVHD response elicited by a second isolated T cell, wherein the second isolated T cell comprises all components of the isolated T cell except it does not comprise the viral protein.

12. A CAR-T cell population comprising: a plurality of the isolated T cell of any one of claims 1 to 11.

13. The CAR-T cell population of claim 12, wherein cell surface expression level of MHC Class I is decreased by at least about 50%, 55%, 60%, 65%, 70%, 75%, 80%, 85%, 90%, 95%, or 99% relative to cell surface expression level of MHC Class I on T cells that do not comprise the viral protein.

14. The CAR-T cell population of claim 13, wherein the cell surface expression level of MHC Class I is measured by flow cytometry.

15. A method of generating an isolated T cell, wherein the method comprises modifying a T-cell expressing a CAR to express a viral protein, wherein the CAR comprises an extracellular ligand-binding domain, a transmembrane domain, and an intracellular signaling domain.

16. The method of claim 14 or 15, further comprising modifying the T cell to express an anti-NK cell antagonist.

17. The method of any one of claims 14 to 16, wherein the viral protein is stably introduced into the cell.

18. The method of any one of claims 14 to 17, wherein a polynucleotide that encodes the viral protein is introduced to the cell by a transposon/transposase system, a viral-based gene transfer system, or electroporation.

19. The method of any one of claims 14 to 18, wherein a polynucleotide that encodes the chimeric antigen receptor is introduced to the cell by a transposon/transposase system, electroporation or a viral-based gene transfer system.

20. The method of claim 19, wherein the viral-based gene transfer system comprises recombinant retrovirus or lentivirus.

21. The method of any one of claims 16 to 20, wherein a polynucleotide that encodes the NK cell antagonist is introduced to the cell by a transposon/transposase system, by viral-based gene transfer system or by electroporation.

22. A pharmaceutical composition comprising the isolated T cell of any one of claims 1 to 11, for use in treating a disorder.

23. The pharmaceutical composition of claim 22, wherein the disorder is cancer, autoimmune disease, or infection.

24. The pharmaceutical composition of claim 22 or 23, wherein the cells are to be provided more than once.

25. The pharmaceutical composition of claim 24, wherein the cells are to be provided to the subject at least about 1, 2, 3, 4, 5, 6, 7, or more days apart.

26. The pharmaceutical composition of claim 25, wherein the disorder is a viral disease, a bacterial disease, a cancer, an inflammatory disease, an immune disease, or an aging-associated disease.

27. A method for treating a disorder in a subject, comprising administering the isolated T cell of any one of claims 1 to 14 to the subject.

28. A method of reducing GVHD in a subject, comprising administering the isolated T cell of any one of claims 1 to 14 to the subject.

29. A method of improving persistence in subject, comprising administering the isolated T cell of any one of claims 1 to 14 to the subject.

30. A method of lengthening the time of a durable response in a subject, comprising administering the isolated T cell of any one of claims 1 to 14 to the subject.

31. The method of any one of claims 27 to 30, wherein the cells are provided to the subject more than once.

32. The method of any one of claims 27 to 31, wherein the subject has been previously treated with a therapeutic agent prior to administration of the isolated T cell.

33. The method of claim 32, wherein the therapeutic agent is an antibody or chemotherapeutic agent.

34. The method of any one of claims 27 to 33, further comprising administering an NK cell antagonist.

35. The method of claim 34, wherein the NK cell antagonist is an anti-NK cell inhibitory receptor antibody.

36. The method of claim 35, wherein the anti-NK cell inhibitory receptor antibody is an anti-KIR antibody.

37. The method of any one of claims 27 to 36, wherein the disorder is a viral disease, a bacterial disease, a cancer, an inflammatory disease, an immune disease, or an aging-associated disease.

38. The method according to claim 37, wherein the cancer is a hematological malignancy or a solid cancer.

39. The method according to claim 38, wherein the hematological malignancy is selected from acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), or multiple myeloma (MM).

40. The method according to claim 38, wherein the solid cancer is selected from biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer.

41. The method of any one of claims 27 to 40, further comprising administering to the subject one or more additional therapeutic agent(s).

42. The method of claim 41, wherein the additional therapeutic agent is an antibody or chemotherapeutic agent.

43. A polynucleotide encoding (i) a viral protein which decreases cell surface expression level of a major histocompatibility complex (MHC) Class I molecule relative to cell surface expression level of MHC Class I molecule of an isolated T cell that does not comprise the viral protein and (ii) a chimeric antigen receptor (CAR).

44. A vector comprising the polynucleotide of claim 43.

45. The vector of claim 44, wherein the vector is a viral vector.


SEQUENCE LISTING (16 sequences)

SEQ ID NO: 1 - protein, 88 aa, Human herpesvirus 1
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 2 - protein, 333 aa, Herpes gammaherpesvirus 8
MEDEDVPVCWICNEELGNERFRACGCTGELENVHRSCLSTWLTISRNTACQICGVVYNTRVVWRPLREMTLLPRLTYQEGLELIVFIFIMTLGAAGLAAATWVWLYIVGGHDPEIDHVAAAAYYVFFVFYQLFVVFGLGAFFHMMRHVGRAYAAVNTRVEVFPYRPRPTSPECAVEEIELQEILPRGDNQDEEGPAGAAPGDQNGPAGAAPGDQDGPADGAPVHRDSEESVDEAAGYKEAGEPTHNDGRDDNVEPTAVGCDCNNLGAERYRATYCGGYVGAQSGDGAYSVSCHNKAGPSSLVDILPQGLPGGGYGSMGVIRKRSAVSSALMFH

SEQ ID NO: 3 - protein, 256 aa, Herpes gammaherpesvirus 8
MASKDVEEGVEGPICWICREEVGNEGIHPCACTGELDVVHPQCLSTWLTVSRNTACQMCRVIYRTRTQWRSRLNLWPEMERQEIFELFLLMSVVVAGLVGVALCTWTLLVILTAPAGTFSPGAVLGFLCFFGFYQIFIVFAFGGICRVSGTVRALYAANNTRVTVLPYRRPRRPTANEDNIELTVLVGPAGGTDEEPTDESSEGDVASGDKERDGSSGDEPDGGPNDRAGLRGTARTDLCAPTKKPVRKNHPKNNG

SEQ ID NO: 4 - protein, 160 aa, Human adenovirus 5
MIRYIILGLLTLASAHGTTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 5 - protein, 186 aa, Human betaherpesvirus 5
MKPVLVLAILAVLFLRLADSVPRPLDVVVSEIRSAHFRVEENQCWFHMGMLYFKGRMSGNFTEKHFVNVGIVSQSYMDRLQVSGEQYHHDERGAYFEWNIGGHPVTHTVDMVDITLSTRWGDPKKYAACVPQVRMDYSSQTINWYLQRSMRDDNWGLLFRTLLVYLFSLVVLVLLTVGVSARLRFI

SEQ ID NO: 6 - protein, 180 aa, Human herpesvirus 5
MDLLIRLGFLLMCALPTPGERSSRDPKTLLSLSPRQQACVPRTKSHRPVCYNDTGDCTDADDSWKQLGEDFAHQCLQAAKKRPKTHKSRPNDRNLEGRLTCQRVRRLLPCDLDIHPSHRLLTLMNNCVCDGAVWNAFRLIERHGFFAVTLYLCCGITLLVVILALLCSITYESTGRGIRR

SEQ ID NO: 7 - protein, 199 aa, Human betaherpesvirus 5
MNNLWKAWVGLWTSMGPLIRLPDGITKAGEDALRPWKSTAKHPWFQIEDNRCYIDNGKLFARGSIVGNMSRFVFDPKADYGGVGENLYVHADDVEFVPGESLKWNVRNLDVMPIFETLALRLVLQGDVIWLRCVPELRVDYTSSAYMWNMQYGMVRKSYTHVAWTIVFYSINITLLVLFIVYVTVDCNLSMMWMRFFVC

SEQ ID NO: 8 - protein, 215 aa, Human betaherpesvirus 5
MNLVMLILALWAPVAGSMPELSLTLFDEPPPLVETEPLPPLSDVSEYRVEYSEARCVLRSGGRLEALWTLRGNLSVPTPTPRVYYQTLEGYADRVPTPVEDVSESLVAKRYWLRDYRVPQRTKLVLFYFSPCHQCQTYYVECEPRCLVPWVPLWSSLEDIERLLFEDRRLMAYYALTIKSAQYTLMMVAVIQVFWGLYVKGWLHRHFPWMFSDQW

SEQ ID NO: 9 - protein, 206 aa, Human immunodeficiency virus type 1
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 10 - protein, 430 aa, Human betaherpesvirus 7
MWTILLFCVPVIYGELYPDFCPLAVVDFDVNATVDDLLLFDISLSKQCSDDKIRHSAVAAMTDNAFFFGNSETQIETDFGKYLAFNCYQVFSTLNHFLFKNFKKTKGLMKRYDKLCLDVESYIHIQIICSPFKSFIRLRRMNETGISPRILETTFYLQNKRNSTWVAIKNYLGEDDPFTYRIWHTLTHAKNFLINSCENDFNQLFFWQRKYLSLAKTFEATFKQGFNPMIEQRNEQRYRTNNIDCSFSKFRQNGVKVAVCKYTGWGVSGFGSLEVLQKIKSPFGEEWKRVGFNSTGAFTPLYGSDVLWGLIFLRVEMTTYVCTCTNKNTGTQIQVTLPDVDLDLLDSEKTSSNVFVDMLCYTLIAILFLAFVTAVVLLGVSCLDGVQKVLTWPLQHIQKEPVSEKIINLTNLMFGQEPLPKKESLKQQCL

SEQ ID NO: 11 - protein, 185 aa, Human betaherpesvirus 5
MLRRGSLRNPLAICLLWWLGVVAAATEETREPTYFTCGCVIQNHVLKGAVKLYGQFPSPKTLRASAWLHDGENHERHRQPILVEGTATATEALYILLPTELSSPEGNRPRNYSATLTLASRDCYERFVCPVYDSGTPMGVLMNLTYLWYLGDYGAILKIYFGLFCGACVITRSLLLICGYYPPRE

SEQ ID NO: 12 - protein, 641 aa, Human herpesvirus 4
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 13 - protein, 60 aa, Human herpesvirus 4
MVHVLERALLEQQSSACGLPGSSTETRPSHPCPEDPDVSRLRLLLVVLCVLFGLLCLLLI

SEQ ID NO: 14 - protein, 87 aa, Human alphaherpesvirus 3
MGSITASFILITMQILFFCEDSSGEPNFAERNFWHASCSARGVYIDGSMITTLFFYASLLGVCVALISLAYHACFRLFTRSVLRSTR

SEQ ID NO: 15 - protein, 225 aa, Cowpox virus
MRSLVIVLLFPSIIYSMVIRRCEKMEEETWKLKIGMCIQAKDFYSKRTDCSVHRPDVGGGLITEGNGYRVVVHDQCEEPNPFIIATTKQTHFGVTHSYIEFSNSNTGAPENIPDCSKHILISVYCDQEASGLDFHTLKYVESNYLHITVKYDTSCINHLGVNYSFMNECERKLTSIYETDTLTCGAKDIQTRDKYLKTCTNTKFDRSVYKTHMQKSKILHVKTEL

SEQ ID NO: 16 - protein, 15 aa, Artificial Sequence
  Made in Lab - linker sequence
GGGGSGGGGSGGGGS